(19) United States
(12) Patent Application Publication
(10) Pub. No.: US 2026/0000486 A1
(43) Pub. Date: Jan. 1, 2026

(54) SYSTEMS AND METHODS FOR ORTHODONTIC APPLIANCE PRODUCTION

(71) Applicant: LUXCREO INC., Belmont, CA (US)

(72) Inventors: Can-ruei YANG, New Taipei (TW); Yisi LU, Ningbo (CN); Mike Shang-Yu YANG, Belmont, CA (US)

(73) Assignee: LUXCREO INC., Belmont, CA (US)

(21) Appl. No.: 19/322,634

(22) Filed: Sep. 8, 2025

Related U.S. Application Data
(63) Continuation of application No. PCT/CN2024/084065, filed on Mar. 27, 2024.
(60) Provisional application No. 63/492,471, filed on Mar. 27, 2023.

(51) Int. Cl. A61C 7/00; A61C 7/08; B29C 64/386; B29L 31/00; B33Y 50/00; B33Y 80/00; G16H 50/50

(52) U.S. Cl. CPC A61C 7/002; A61C 7/08; B29C 64/386; B33Y 50/00; B33Y 80/00; G16H 50/50; B29L 2031/753

(57) ABSTRACT

The present disclosure provides systems and methods for orthodontic appliance production. The methods may include determining a baseline thickness of an orthodontic appliance for orthodontically treating teeth of a target subject. The methods may include, for each local point on the teeth of the target subject, determining a movement path of the local point from an initial location of the local point to a target location of the local point. The methods may also include, for each local point on the teeth of the target subject, determining, based on the movement path and the baseline thickness, a target thickness of the orthodontic appliance corresponding to the local point. The methods may further include directing a three-dimensional (3D) printer to integrally produce, based on the target thickness of the orthodontic appliance at each local point, the orthodontic appliance.

### CROSS-REFERENCE TO RELATED APPLICATIONS

This application is a continuation of International Application No. PCT/CN2024/084065, filed on Mar. 27, 2024, which claims priority to U.S. Provisional Patent Application No. 63/492,471, filed on Mar. 27, 2023, the contents of which are hereby incorporated by reference.

### TECHNICAL FIELD

The present disclosure generally relates to the field of three-dimensional (3D) printing, and more particularly, relates to systems and methods for orthodontic appliance production through 3D printing techniques.

### BACKGROUND

3D printing techniques have been widely used in the orthodontic market, such as in the production process of orthodontic appliances (e.g., aligners, retainers, etc.). However, in the current production process, the orthodontic appliances are not integrally produced. In other words, only a portion of components of the orthodontic appliances are produced using the 3D printing techniques. Alternatively, the components of the orthodontic appliances are printed respectively, and assembled to form the orthodontic appliances, which are cumbersome and wasteful. In addition, the current production process includes a thermoforming operation that uses uniform thermoplastic sheets. The orthodontic appliances made of the uniform thermoplastic sheets also have uniform thickness and can not provide customized and localized forces to different teeth or different local points on the teeth.

Therefore, it is desirable to provide systems and methods for orthodontic appliance production, which can integrally produce the orthodontic appliances with customized and ununiform thicknesses, thereby providing customized forces to the teeth and improving the orthodontic effectiveness.

### SUMMARY

In an aspect of the present disclosure, a method for orthodontic appliance production is provided. The method may be implemented on a computing device having at least one processor and at least one storage device. The methods may include determining a baseline thickness of an orthodontic appliance for orthodontically treating teeth of a target subject. The methods may include, for each local point on the teeth of the target subject, determining a movement path of the local point from an initial location of the local point to a target location of the local point. The methods may also include, for each local point on the teeth of the target subject, determining, based on the movement path and the baseline thickness, a target thickness of the orthodontic appliance corresponding to the local point. The methods may further include directing a three-dimensional (3D) printer to integrally produce, based on the target thickness of the orthodontic appliance at each local point, the orthodontic appliance.

In another aspect of the present disclosure, a system orthodontic appliance production is provided. The system may include at least one storage device including a set of instructions and at least one processor configured to communicate with the at least one storage device. When executing the set of instructions, the at least one processor may be configured to direct the system to perform following operations. The operations may include determining a baseline thickness of an orthodontic appliance for orthodontically treating teeth of a target subject. The operations may include, for each local point on the teeth of the target subject, determining a movement path of the local point from an initial location of the local point to a target location of the local point. The operations may also include, for each local point on the teeth of the target subject, determining, based on the movement path and the baseline thickness, a target thickness of the orthodontic appliance corresponding to the local point. The operations may further include directing a three-dimensional (3D) printer to integrally produce, based on the target thickness of the orthodontic appliance at each local point, the orthodontic appliance.

In still another aspect of the present disclosure, a non-transitory computer readable medium is provided. The medium may include executable instructions that, when executed by at least one processor, direct the at least one processor to perform a method for orthodontic appliance production. The methods may include determining a baseline thickness of an orthodontic appliance for orthodontically treating teeth of a target subject. The methods may include, for each local point on the teeth of the target subject, determining a movement path of the local point from an initial location of the local point to a target location of the local point. The methods may also include, for each local point on the teeth of the target subject, determining, based on the movement path and the baseline thickness, a target thickness of the orthodontic appliance corresponding to the local point. The methods may further include directing a three-dimensional (3D) printer to integrally produce, based on the target thickness of the orthodontic appliance at each local point, the orthodontic appliance.

Additional features will be set forth in part in the description which follows, and in part will become apparent to those skilled in the art upon examination of the following and the accompanying drawings or may be learned by production or operation of the examples. The features of the present disclosure may be realized and attained by practice or use of various aspects of the methodologies, instrumentalities, and combinations set forth in the detailed examples discussed below.

### BRIEF DESCRIPTION OF THE DRAWINGS

The present disclosure is further described in terms of exemplary embodiments. These exemplary embodiments are described in detail with reference to the drawings. These embodiments are non-limiting exemplary embodiments, in which like reference numerals represent similar structures throughout the several views of the drawings, and wherein:

FIG. 1 is a schematic diagram illustrating an exemplary system for orthodontic appliance production according to some embodiments of the present disclosure;

FIG. 2 is a block diagram illustrating an exemplary processing device according to some embodiments of the present disclosure;

FIG. 3 is a flowchart illustrating an exemplary process for orthodontic appliance production according to some embodiments of the present disclosure;

FIG. 4A is a schematic diagram illustrating an exemplary digital tooth model according to some embodiments of the present disclosure;

FIG. 4B is a schematic diagram illustrating an exemplary preliminary digital model of an orthodontic appliance according to some embodiments of the present disclosure;

FIG. 5 is a schematic diagram illustrating an exemplary process for orthodontic treatment according to some embodiments of the present disclosure;

FIG. 6 is a flowchart illustrating an exemplary process for determining a target thickness of an orthodontic appliance at a local point according to some embodiments of the present disclosure;

FIG. 7 is a schematic diagram illustrating an exemplary process for determining a target thickness of an orthodontic appliance at a local point using a thickness determination model according to some embodiments of the present disclosure;

FIG. 8 is a schematic diagram illustrating an exemplary process for orthodontic appliance production according to some embodiments of the present disclosure; and

FIG. 9 is a schematic diagram illustrating an exemplary computing device according to some embodiments of the present disclosure.

### DETAILED DESCRIPTION

In the following detailed description, numerous specific details are set forth by way of examples in order to provide a thorough understanding of the relevant disclosure. However, it should be apparent to those skilled in the art that the present disclosure may be practiced without such details. In other instances, well-known methods, procedures, systems, components, and/or circuitry have been described at a relatively high level, without detail, in order to avoid unnecessarily obscuring aspects of the present disclosure. Various modifications to the disclosed embodiments will be readily apparent to those skilled in the art, and the general principles defined herein may be applied to other embodiments and applications without departing from the spirit and scope of the present disclosure. Thus, the present disclosure is not limited to the embodiments shown, but to be accorded the widest scope consistent with the claims.

The terminology used herein is for the purpose of describing particular example embodiments only and is not intended to be limiting. As used herein, the singular forms “a,” “an,” and “the” may be intended to include the plural forms as well, unless the context clearly indicates otherwise. It will be further understood that the terms “comprise,” “comprises,” and/or “comprising,” “include,” “includes,” and/or “including,” when used in this specification, specify the presence of stated features, integers, steps, operations, elements, and/or components, but do not preclude the presence or addition of one or more other features, integers, steps, operations, elements, components, and/or groups thereof.

It will be understood that when a unit, engine, module, or block is referred to as being “on,” “connected to,” or “coupled to,” another unit, engine, module, or block, it may be directly on, connected or coupled to, or communicate with the other unit, engine, module, or block, or an intervening unit, engine, module, or block may be present, unless the context clearly indicates otherwise. As used herein, the term “and/or” includes any and all combinations of one or more of the associated listed items.

These and other features, and characteristics of the present disclosure, as well as the methods of operation and functions of the related elements of structure and the combination of parts and economics of manufacture, may become more apparent upon consideration of the following description with reference to the accompanying drawings, all of which form a part of this disclosure. It is to be expressly understood, however, that the drawings are for the purpose of illustration and description only and are not intended to limit the scope of the present disclosure. It is understood that the drawings are not to scale.

As mentioned above, 3D printing techniques have been widely used in the orthodontic field. For example, invisible aligners can be produced using the 3D printing techniques. The invisible aligners are used for orthodontic treatment on teeth of a target subject. At the beginning of the orthodontic treatment, a doctor may determine an orthodontic treatment plan to move the teeth of the target subject from initial locations to desired locations (or target locations). The orthodontic treatment plan may include a plurality of treatment steps. At each treatment step, a corresponding clear aligner is used to move the teeth to pre-determined locations by applying controlled forces to local points on the teeth. Each aligner is designed to be worn for 1-2 weeks and move the teeth in small increments. For instance, the aligner applies the controlled forces on the teeth so that the teeth move to match the aligner. When a new aligner is worn, the teeth are slightly more aligned than the teeth used to be. As each aligner is replaced with a next aligner, the teeth continue to move until they have moved to their desired locations.

At present, the aligners are produced according to the following operations. In operation 1, a dental model file of the target subject is obtained by performing a scan of a physical impression or an intraoral scan on the target subject. In operation 2, the orthodontic treatment plan is determined using an orthodontic software, and a corresponding dental model for each treatment step is generated. In operation 3, the dental models for all the treatment steps are printed out using the 3D printing techniques. In operation 4, the aligners are produced by thermoforming the dental models for all the treatment steps. In operation 5, the aligners are post-processed, such as trimmed, polished, etc.

The performance of an aligner to move the teeth depends on many variables, one of which is a thickness of the aligner. The thickness influences the amount of the force and/or torque that can be applied onto the teeth. If the aligner is produced using the thermoforming operation and the thermoforming operation uses uniform thermoplastic sheets, the aligner may have uniform thicknesses and can not provide customized and localized forces to different teeth or the local points on the teeth, which reduces the orthodontic effectiveness. During the use of the orthodontic aligner, certain areas may require localized thickening due to functional requirements. For example, some areas of the aligner may need to enhance gripping forces on the teeth. In traditional thermoformed aligners, this issue is addressed by adding attachments. Additionally, a solution for increasing the gripping forces in the traditional thermoformed aligners involves adding special structures, such as a power ridge structure of Invisalign®. Moreover, during the process of removing the aligner, if the aligner fits relatively well with the teeth and is relatively thin, no appropriate points may be disposed on the aligner for applying force to remove the aligner.

In order to solve the above problems, the present disclosure provides systems and methods for orthodontic appliance production. The methods may include determining a baseline thickness of an orthodontic appliance for orthodontically treating teeth of a target subject. For each local point on the teeth of the target subject, the methods may include determining a movement path of the local point from an initial location of the local point to a target location of the local point, and determining a target thickness of the orthodontic appliance corresponding to the local point based on the movement path and the baseline thickness. The methods may further include directing a 3D printer to integrally produce the orthodontic appliance based on the target thickness of the orthodontic appliance at each local point. By determining the target thickness of the orthodontic appliance at each local point, the orthodontic appliance with customized thicknesses can be integrally produced using the 3D printing techniques, which can provide the customized and localized forces and/or torque to different teeth or the local points on the teeth, thereby improving the orthodontic effectiveness.

In addition, correction thicknesses (e.g., a first correction thickness, a second correction thickness, a third correction thickness, a fourth correction thickness, etc.) are introduced to determine the target thickness of the orthodontic appliance at each local point, which combines feature information of the local points and/or the teeth, thereby improving the accuracy of the thickness determination, the customization of the orthodontic appliance, and the orthodontic effectiveness. At the same time, the target thickness can be directly determined based on the baseline thickness and the correction thickness, so that the orthodontic appliance is integrally produced, and no attachments or less attachments (e.g., attachments for improving the gripping forces between the teeth and the orthodontic appliance, attachments for providing forces to easily remove the orthodontic appliance from the teeth, etc.) need to be disposed on the orthodontic appliance, which can simplify the orthodontic appliance production process, thereby improving the efficiency of the orthodontic appliance production. Moreover, the user experience can be improved through the correction thicknesses. For example, by introducing the third correction thickness, focal points on the orthodontic appliance corresponding to at the lingual side of the posterior molars can be designed, which can provide the forces to easily remove the orthodontic appliance from the teeth. At the same time, a portion of the orthodontic appliance corresponding to the lingual side of the posterior molars can be thickened, which can prevent deformation and fracture of the orthodontic appliance.

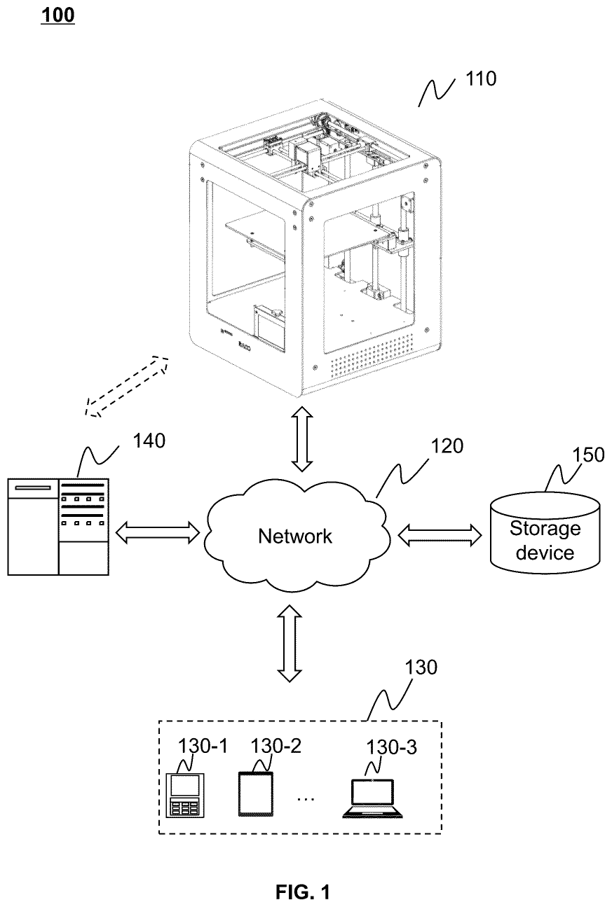

FIG. 1 is a schematic diagram illustrating an exemplary system 100 for orthodontic appliance production according to some embodiments of the present disclosure.

As illustrated in FIG. 1, the system 100 for orthodontic appliance production may include a 3D printer 110, a network 120, at least one terminal device 130, a processing device 140, and a storage device 150. The components of the system 100 may be connected in one or more of various ways. For example, the 3D printer 110 may be connected to the processing device 140 through the network 120. As another example, the 3D printer 110 may be connected to the processing device 140 directly (as indicated by the bi-directional arrow in dotted lines linking the 3D printer 110 and the processing device 140).

The 3D printer 110 may be configured to produce a 3D object (e.g., an orthodontic appliance) by processing printing materials based on instructions from the processing device 140. For example, the 3D printer 110 may successively stack the printing materials layer by layer based on a predetermined printing model of the 3D object. In some embodiments, the printing material may include a plastic material, a resin material, a metal material, a rubber material, a wax material, or the like, or any combination thereof. For example, the printing materials may be a resin material including thermal-curing components and photocurable components. The photocurable components may be cured by light beams and the thermal-curing components may be cured in a heating process. In some embodiments, the 3D printer 110 may be any type of printer. Exemplary types may include a digital light processing (DLP) printer, a liquid crystal display (LCD) printer, a stereo lithography (SLA) printer, a polymer jetting printer, a selective laser sintering (SLS) printer, a selective laser melting (SLM) printer, an electron beam melting (EBM) printer, a fused deposition modeling (FDM) printer, a layer laminate manufacturing (LLM) printer, an aerosol printer, a bioplotter printer, or the like, or any combination thereof.

The network 120 may include any suitable network that can facilitate the exchange of information and/or data for the system 100. In some embodiments, one or more components (e.g., the 3D printer 110, the at least one terminal device 130, the processing device 140, the storage device 150) of the system 100 may communicate with one or more other components of the system 100 via the network 120. In some embodiments, the network 120 may be any type of wired or wireless network, or a combination thereof. Merely by way of example, the network 120 may include a cable network, a wireline network, a fiber-optic network, a telecommunications network, an intranet, a wireless local area network (WLAN), a metropolitan area network (MAN), a public telephone switched network (PSTN), a Bluetooth™M network, a ZigBee™ network, a near field communication (NFC) network, or the like, or any combination thereof. In some embodiments, the network 120 may include one or more network access points. For example, the network 120 may include wired and/or wireless network access points such as base stations and/or internet exchange points through which one or more components of the system 100 may be connected to the network 120 to exchange data and/or information.

The at least one terminal device 130 may include a mobile device 130-1, a tablet computer 130-2, a laptop computer 130-3, or the like, or any combination thereof. In some embodiments, the mobile device 130-1 may include a smart home device, a wearable device, a smart mobile device, a virtual reality device, an augmented reality device, or the like, or any combination thereof. In some embodiments, the smart home device may include a smart lighting device, a smart control device of an intelligent electrical apparatus, a smart monitoring device, a smart television, a smart video camera, a smart interphone, or the like, or any combination thereof. In some embodiments, the wearable device may include a smart bracelet, smart footgear, a pair of smart glasses, a smart helmet, a smart watch, smart clothing, a smart backpack, a smart accessory, or the like, or any combination thereof. In some embodiments, the smart mobile device may include a smartphone, a personal digital assistant (PDA), a gaming device, a navigation device, a point of sale (POS) device, or the like, or any combination thereof. In some embodiments, the virtual reality device and/or the augmented reality device may include a virtual reality helmet, a virtual reality glass, a virtual reality patch, an augmented reality helmet, an augmented reality glass, an augmented reality patch, or the like, or any combination thereof. For example, the virtual reality device and/or the augmented reality device may include a Google™ Glass, an Oculus Rift, a Hololens, a Gear VR, etc.

In some embodiments, the 3D printer 110 and/or the processing device 140 may be remotely operated through the at least one terminal device 130. In some embodiments, the 3D printer 110 and/or the processing device 140 may be operated through the at least one terminal device 130 via a wireless connection. In some embodiments, the at least one terminal device 130 may receive information and/or instructions inputted by a user and send the received information and/or instructions to the 3D printer 110 or the processing device 140 via the network 120. In some embodiments, the at least one terminal device 130 may receive data and/or information from the processing device 140. In some embodiments, the at least one terminal 130 may provide a user interface via which a user may view information and/or input data and/or instructions to the system 100. For example, the at least one terminal 130 may include a display that can display information in a human-readable form, such as text, image, audio, video, graph, animation, or the like, or any combination thereof. The display of the at least one terminal 130 may include a cathode ray tube (CRT) display, a liquid crystal display (LCD), a light-emitting diode (LED) display, a plasma display panel (PDP), a three-dimensional (3D) display, or the like, or any combination thereof. In some embodiments, the at least one terminal device 130 may be part of the processing device 140. In some embodiments, the at least one terminal device 130 may be omitted.

The processing device 140 may process data and/or information obtained from the 3D printer 110, the at least one terminal device 130, and/or the storage device 150. For example, the processing device 140 may determine a baseline thickness of the orthodontic appliance for orthodontically treating teeth of a target subject. As another example, for each local point on the teeth of the target subject, the processing device 140 may determine a movement path of the local point from an initial location of the local point to a target location of the local point, and determine a target thickness of the orthodontic appliance corresponding to the local point based on the movement path and the baseline thickness. As still another example, the processing device 140 may direct the 3D printer 110 to integrally produce the orthodontic appliance based on the target thickness of the orthodontic appliance at each local point.

In some embodiments, the processing device 140 may be a single server or a server group. The server group may be centralized or distributed. In some embodiments, the processing device 140 may be local or remote. For example, the processing device 140 may access information and/or data stored in or acquired by the 3D printer 110, the at least one terminal device 130, and/or the storage device 150 via the network 120. As another example, the processing device 140 may be directly connected to the 3D printer 110, the at least one terminal device 130, and/or the storage device 150 to access stored or acquired information and/or data. In some embodiments, the processing device 140 may be implemented on a cloud platform. Merely by way of example, the cloud platform may include a private cloud, a public cloud, a hybrid cloud, a community cloud, a distributed cloud, an inter-cloud, a multi-cloud, or the like, or any combination thereof. In some embodiments, the processing device 140 may be integrated into the 3D printer 110.

The storage device 150 may store data and/or instructions. In some embodiments, the storage device 150 may store data obtained from the 3D printer 110, the at least one terminal device 130, and/or the processing device 140. For example, the storage device 150 may store the baseline thickness of the orthodontic appliance, the movement path corresponding to each local point, the target thickness of the orthodontic appliance at each local point, etc. In some embodiments, the storage device 150 may store data and/or instructions that the processing device 140 may execute or use to perform exemplary methods described in the present disclosure. For example, the storage device 150 may store instructions that the processing device 140 may execute to treating a printed model. In some embodiments, the storage device 150 may include a mass storage device, a removable storage device, a volatile read-and-write memory, a read-only memory (ROM), or the like, or any combination thereof. In some embodiments, the storage device 150 may be implemented on a cloud platform. Merely by way of example, the cloud platform may include a private cloud, a public cloud, a hybrid cloud, a community cloud, a distributed cloud, an inter-cloud, a multi-cloud, or the like, or any combination thereof.

In some embodiments, the storage device 150 may be connected to the network 120 to communicate with one or more components (e.g., the 3D printer 110, the processing device 140, the at least one terminal device 130) of the system 100. One or more components of the system 100 may access the data or instructions stored in the storage device 150 via the network 120. In some embodiments, the storage device 150 may be directly connected to or communicate with one or more components (e.g., the 3D printer 110, the processing device 140, the at least one terminal device 130) of the system 100. In some embodiments, the storage device 150 may be part of the processing device 140.

In some embodiments, the system 100 may further include other component(s) (e.g., one or more power supplies, a 3D scanner, etc.) connected to one or more components (e.g., the 3D printer 110, the processing device 140, the at least one terminal device 130, the storage device 150) of the system 100.

It should be noted that the above description is merely provided for the purposes of illustration, and is not intended to limit the scope of the present disclosure. For persons having ordinary skills in the art, multiple variations and modifications may be made under the teachings of the present disclosure. Features, structures, methods, and other characteristics of the exemplary embodiments described herein may be combined in various ways to obtain additional and/or alternative exemplary embodiments. However, those variations and modifications do not depart from the scope of the present disclosure.

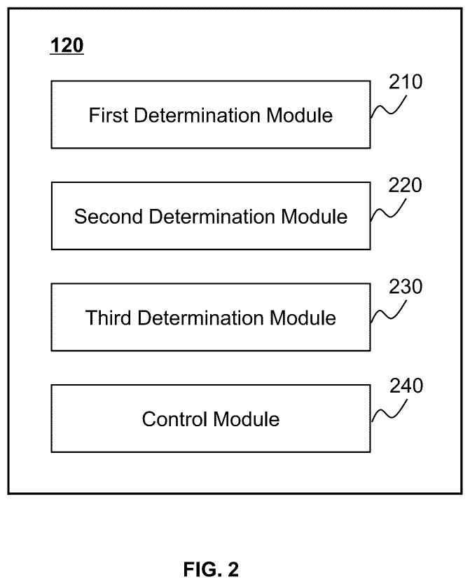

FIG. 2 is a block diagram illustrating an exemplary processing device 140 according to some embodiments of the present disclosure. In some embodiments, the processing device 140 may be in communication with a computer-readable storage medium (e.g., the storage device 150 illustrated in FIG. 1) and may execute instructions stored in the computer-readable storage medium. The processing device 140 may include a first determination module 210, a second determination module 220, a third determination module 230, and a control module 240.

The first determination module 210 may be configured to determine a baseline thickness of an orthodontic appliance for orthodontically treating teeth of a target subject. The baseline thickness refers to a preliminarily determined thickness of the orthodontic appliance. In some embodiments, the baseline thickness of the orthodontic appliance at each and every local point on the teeth of the target subject may be the same. More descriptions regarding the determination of the baseline thickness of the orthodontic appliance may be found elsewhere in the present disclosure. See, e.g., operation 302 and relevant descriptions thereof.

The second determination module 220 may be configured to, for each local point on the teeth of the target subject, determine a movement path of the local point from an initial location of the local point to a target location of the local point. A local point refers to a point located on a surface of the teeth of the target subject. The initial location of a local point refers to a location of the local point before using the orthodontic appliance, and the target location of the local point refers to a desired location of the local point after using the orthodontic appliance. Correspondingly, the movement path of the local point refers to a predicted path along which the local point moves from the initial location to the target location during the orthodontic treatment applied by the orthodontic appliance. More descriptions regarding the determination of the movement path of the local point may be found elsewhere in the present disclosure. See, e.g., operation 304 and relevant descriptions thereof.

The third determination module 230 may be configured to, for each local point on the teeth of the target subject, determine a target thickness of the orthodontic appliance corresponding to the local point based on the movement path and the baseline thickness. The orthodontic appliance to be produced may include a point corresponding to each local point on the teeth of the target subject, and the point may cover the corresponding local point when the orthodontic appliance is worn on the target subject. The target thickness corresponding to a local point refers to a desired thickness of the orthodontic appliance at the corresponding point of the local point. More descriptions regarding the determination of the target thickness of the orthodontic appliance corresponding to the local point may be found elsewhere in the present disclosure. See, e.g., operation 306 and relevant descriptions thereof.

The control module 240 may be configured to direct a 3D printer to integrally produce, based on the target thickness of the orthodontic appliance at each local point, the orthodontic appliance. More descriptions regarding the production of the orthodontic appliance may be found elsewhere in the present disclosure. See, e.g., operation 308 and relevant descriptions thereof.

It should be noted that the above descriptions of the processing device 140 are provided for the purposes of illustration, and not intended to limit the scope of the present disclosure. For persons having ordinary skills in the art, various variations and modifications may be conducted under the guidance of the present disclosure. However, those variations and modifications do not depart from the scope of the present disclosure. In some embodiments, the processing device 140 may include one or more other modules. For example, the processing device 140 may include a storage module to store data generated by the modules in the processing device 140. In some embodiments, any two of the modules may be combined as a single module, and any one of the modules may be divided into two or more units. For example, the first determination module 210, the second determination module 220, and the third determination module 230 may be combined as a single determination module.

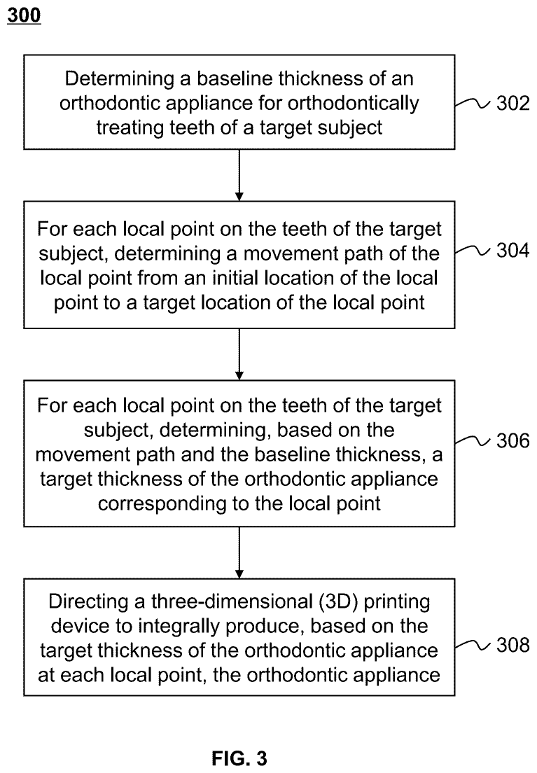

FIG. 3 is a flowchart illustrating an exemplary process 300 for orthodontic appliance production according to some embodiments of the present disclosure.

In 302, the processing device 140 (e.g., the first determination module 210) may determine a baseline thickness of an orthodontic appliance for orthodontically treating teeth of a target subject.

The target subject refers to a subject whose teeth need to be orthodontically treated. For example, the target subject may include a teenager whose teeth are irregular.

The orthodontic appliance refers to a dental appliance for the treatment of malocclusion, which is caused by tooth irregularity (e.g., overcrowded or misaligned teeth), disproportionate jaw relationships, or a combination thereof. Exemplary orthodontic appliances may include an active orthodontic appliance, a passive orthodontic appliance, a functional orthodontic appliance, or the like, or any combination thereof. The active orthodontic appliance refers to a device used to apply forces to the teeth to change the placement and/or the relationship of the teeth. For example, the active orthodontic appliance may include an aligner, a brace, a headgear, an expander, or the like, or any combination thereof. The passive orthodontic appliance refers to a device that relies on a bite of the target subject for tooth movement(s). For example, the passive orthodontic appliance may include a retainer, a night guard, or the like, or any combination thereof. The functional orthodontic appliance refers to a device that uses a muscle action of the target subject and/or responses of a nervous system of the target subject to generate orthodontic or orthopaedic forces. For example, the functional orthodontic appliance may include an orthodontic headgear, a herbst appliance, a twin block appliance, a fixed lingual mandibular growth modificator (FLMGM), or the like, or any combination thereof.

In order to orthodontically treat the teeth of the target subject, an orthodontic treatment plan including a plurality of treatment steps may be determined, and at least one of the plurality of treatment steps may correspond to an orthodontic appliance. For illustration purposes, one of the at least one orthodontic appliance is described herein, and the orthodontic appliance corresponds to one treatment step in the orthodontic treatment plan.

The baseline thickness refers to a preliminarily determined thickness of the orthodontic appliance. In some embodiments, the baseline thickness of the orthodontic appliance at each and every local point on the teeth of the target subject may be the same. In some embodiments, the baseline thickness of the orthodontic appliance may be at a millimeter level. For example, the baseline thickness of the orthodontic appliance may be within a range from 0.1 millimeters (mm) to 5.0 mm. As another example, the baseline thickness of the orthodontic appliance may be within a range from 0.1 mm to 1.0 mm. As still another example, the baseline thickness of the orthodontic appliance may be within a range from 0.2 mm to 0.8 mm. As yet another example, the baseline thickness of the orthodontic appliance may be within a range from 0.3 mm to 0.7 mm. As yet another example, the baseline thickness of the orthodontic appliance may be within a range from 0.4 mm to 0.6 mm. As yet another example, the baseline thickness of the orthodontic appliance may be 0.5 mm.

In some embodiments, the processing device 140 may determine the baseline thickness of the orthodontic appliance based on an empirical value. For example, the processing device 140 may determine the empirical value based on historical data, and designate the empirical value as the baseline thickness of the orthodontic appliance. In some embodiments, the processing device 140 may determine the baseline thickness of the orthodontic appliance based on an input of a user (e.g., a doctor). For example, the baseline thickness may be input by the user via a user terminal.

In some embodiments, the processing device 140 may determine the baseline thickness of the orthodontic appliance based on a 3D printer used for producing the orthodontic appliance. For example, if an optimal printing thickness of the 3D printer 110 is 0.5 mm, the processing device 140 may determine 0.5 mm as the baseline thickness of the orthodontic appliance.

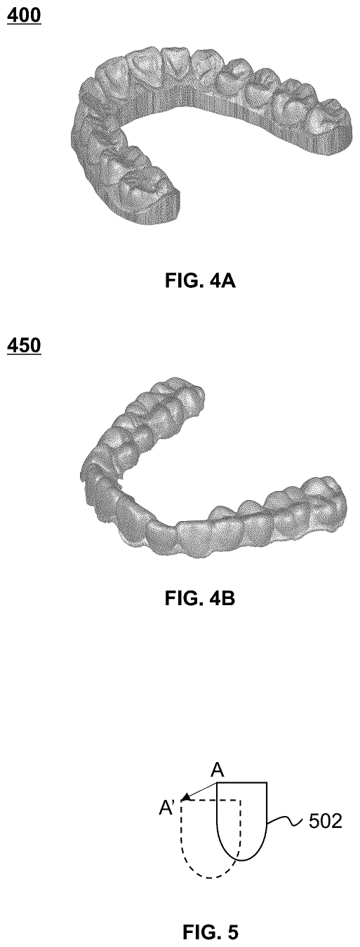

In some embodiments, the processing device 140 may determine the baseline thickness of the orthodontic appliance based on the condition of the teeth of the target subject. For example, an image of the teeth of the target subject may be acquired by scanning the teeth of the target subject (e.g., performing a scan of a physical impression or an intraoral scan on the teeth of the target subject), and the processing device 140 may determine the baseline thickness of the orthodontic appliance based on the image of the teeth of the target subject. Referring to FIGS. 4A and 4B, a digital tooth model 400 corresponding to the teeth of the target subject may be generated based on the image of the teeth of the target subject, and a preliminary digital model 450 of the orthodontic appliance may be generated based on the digital tooth model 400. Correspondingly, a baseline thickness of the orthodontic appliance may be determined based on the preliminary digital model 450 of the orthodontic appliance. In some embodiments, the digital tooth model 400 and/or the preliminary digital model 450 may be generated using computer software, such as LuxCreo's LuxDesign.

In some embodiments, the processing device 140 may determine the baseline thickness of the orthodontic appliance based on the orthodontic treatment plan corresponding to the target subject. For example, the orthodontic treatment plan corresponding to the target subject may be pre-determined, and the processing device 140 may determine the baseline thickness of the orthodontic appliance based on the orthodontic treatment plan (e.g., each treatment step in the orthodontic treatment plan) corresponding to the target subject. In some embodiments, the baseline thickness of the orthodontic appliance corresponding to each treatment step in the orthodontic treatment plan may be the same or different.

In 304, for each local point on the teeth of the target subject, the processing device 140 (e.g., the second determination module 220) may determine a movement path of the local point from an initial location of the local point to a target location of the local point.

A local point refers to a point located on a surface of the teeth of the target subject. For example, the local points may include points located on dental enamels of the teeth of the target subject.

The initial location of a local point refers to a location of the local point before using the orthodontic appliance, and the target location of the local point refers to a desired location of the local point after using the orthodontic appliance. Correspondingly, the movement path of the local point refers to a predicted path along which the local point moves from the initial location to the target location during the orthodontic treatment applied by the orthodontic appliance. For example, FIG. 5 is a schematic diagram illustrating an exemplary moving path of a local point according to some embodiments of the present disclosure. As illustrated in FIG. 5, during the orthodontic treatment process, a local point at a top-left corner of a tooth 502 moves from an initial location A to a target location A′, and a corresponding movement path of the local point is indicated by an arrow AA′.

In some embodiments, the processing device 140 may determine the movement path of each local point based on the orthodontic treatment plan. For example, the orthodontic treatment plan (e.g., each treatment step in the orthodontic treatment plan) may include the initial location of each local point and the target location of each local point. For instance, the orthodontic treatment plan may include a first digital tooth model corresponding to the current teeth and a second digital tooth model corresponding to target teeth after the orthodontic treatment (or a treatment step in the orthodontic treatment). The processing device 140 may determine a corresponding relationship between local points in the first digital tooth model and the second digital tooth model, and determine the movement path of each local point based on the corresponding relationship, the first digital tooth model, and the second digital tooth model. For example, the moving path of a local point may be determined based on the position of the local point in the first digital tooth model and the position of the corresponding local point in the second digital tooth model.

In some embodiments, the movement path may be represented by a vector. In some embodiments, the movement path may be represented by parameters such as a length of the movement path, a direction of the movement path, positions of points along the movement path, or the like, or any combination thereof.

In some embodiments, a length of the movement path of each local point may not exceed a length threshold. The length threshold may be equal to a maximum distance between the initial location and the target location of each local point. In some embodiments, the length threshold may be determined based on a maximum thickness of the orthodontic appliance. Since the orthodontic appliance provides forces for orthodontically treating the teeth of the target subject, the maximum thickness of the orthodontic appliance may determine the length threshold. For example, the length threshold may be 1 mm, and the length of the movement path of each local point may be within a range from 0 mm to 1 mm. As another example, the length threshold may be 0.4 mm, and the length of the movement path of each local point may be within a range from 0 mm to 0.4 mm. As still another example, the length threshold may be 0.3 mm, and the length of the movement path of each local point may be within a range from 0 mm to 0.3 mm.

In 306, for each local point on the teeth of the target subject, the processing device 140 (e.g., the third determination module 230) may determine, based on the movement path and the baseline thickness, a target thickness of the orthodontic appliance corresponding to the local point.

The orthodontic appliance to be produced may include a point corresponding to each local point on the teeth of the target subject, and the point may cover the corresponding local point when the orthodontic appliance is worn on the target subject. The target thickness corresponding to a local point refers to a desired thickness of the orthodontic appliance at the corresponding point of the local point.

In some embodiments, for each local point on the teeth of the target subject, the processing device 140 may determine the target thickness of the orthodontic appliance corresponding to the local point by constructing a function relating to the movement path and the baseline thickness. For example, the target thickness of the orthodontic appliance corresponding to the local point may be determined according to Equation (1):

$$T = {kT}_{b} , \qquad ( 1 )$$

where T refers to the target thickness of the orthodontic appliance corresponding to the local point; T_{b} refers to the baseline thickness of the orthodontic appliance; and k refers to a coefficient determined based on the movement path. For example, k is positively correlated with the length of the movement path.

As another example, the target thickness of the orthodontic appliance corresponding to the local point may be determined according to Equation (2):

$$T = tP , \qquad ( 2 )$$

where P refers to the length of the movement path; and t refers to a coefficient determined based on the baseline thickness of the orthodontic appliance. For example, t is positively correlated with the baseline thickness of the orthodontic appliance.

In some embodiments, for each local point on the teeth of the target subject, the processing device 140 may determine a first correction thickness of the orthodontic appliance corresponding to the local point based on the movement path, and determine the target thickness of the orthodontic appliance corresponding to the local point based on the first correction thickness and the baseline thickness. For example, the target thickness of the orthodontic appliance corresponding to the local point may be determined according to Equation (3):

$$T = {T}_{b} + {T}_{fc} , \qquad ( 3 )$$

where T_{fc} refers to the first correction thickness of the orthodontic appliance corresponding to the local point.

The first correction thickness may relate to a component of the movement path along a normal direction of the local point. The normal direction of the local point refers to the normal direction of the surface of the teeth at the local point. For example, the first correction thickness may be equal to the component of the movement path along the normal direction of the local point. For instance, the processing device 140 may determine a local plane where the local point is located, and determine a direction perpendicular to the local plane at the local point as the normal direction of the local point. As another example, the processing device 140 may obtain a digital tooth model (e.g., the digital tooth model 400) of the teeth of the target subject. The processing device 140 may determine a surface equation of the teeth at the local point based on the digital tooth model, and determine the normal direction by determining a gradient of the surface equation. Then, the processing device 140 may determine the component of the movement path along the normal direction of the local point, and designate the length of the component of the movement path along the normal direction of the local point as the first correction thickness.

Merely by way of example, referring to FIG. 5, assuming that the baseline thickness of the orthodontic appliance is 0.5 mm, and the length of the movement path AA′ is 0.3 mm, if the normal direction of the local point is parallel to the movement path AA′, the length of the component of the movement path along the normal direction of the local point may be 0.3 mm, and the first correction thickness may also be 0.3 mm. Therefore, the target thickness of the orthodontic appliance corresponding to the local point may be 0.8 mm (i.e., a sum of 0.5 mm and 0.3 mm) according to Equation (3). If the normal direction of the local point is perpendicular to the movement path, the length of the component of the movement path along the normal direction of the local point may be 0 mm, and the first correction thickness may also be 0 mm. Therefore, the target thickness of the orthodontic appliance corresponding to the local point may be 0.5 mm (i.e., a sum of 0.5 mm and 0 mm) according to Equation (3).

In some embodiments, the first correction thickness may relate to component(s) of the movement path along one or more reference directions corresponding to the normal direction of the local point. For example, the processing device 140 may determine the one or more reference directions based on the normal direction of the local point. For each of the one or more reference directions, the processing device 140 may determine a component of the movement path along the direction. The processing device 140 may further determine the first correction thickness of the orthodontic appliance corresponding to the local point based on the component of the movement path along the normal direction and the component of the movement path along each reference direction.

A reference direction refers to a direction around the normal direction. For example, an included angle between the reference direction and the normal direction may not exceed an angle threshold. The angle threshold may be determined based on a system default setting, or set manually by the user. For example, the angle threshold may be 1 degree, 5 degrees, 10 degrees, 15 degrees, 20 degrees, 30 degrees, etc.

In some embodiments, the processing device 140 may determine the one or more reference directions further based on a curvature of the teeth at the local point and/or an angle between the movement path of the local point and the normal direction of the local point. The curvature of the teeth at the local point may reflect a degree of curvature of the teeth at the local point. For example, a curvature of a local point at a corner of a tooth may be larger than a curvature of a local point at a surface of the tooth. In some embodiments, the curvature of the teeth at the local point may be determined based on the digital tooth model of the teeth or the surface equation at the local point. The angle between the movement path of the local point and the normal direction of the local point may reflect a degree of curvature of the movement path of the local point during the orthodontic treatment.

In some embodiments, the larger the curvature of the teeth at the local point and/or the angle between the movement path of the local point and the normal direction of the local point, the larger the angle threshold and a count of the one or more reference directions. For example, if the curvature of the teeth at the local point is larger than or equal to a curvature threshold (e.g., 90 degrees), the processing device 140 may determine the angle threshold as 30 degrees and the count of the one or more reference directions as 8. As another example, if the curvature of the teeth at the local point is less than the curvature threshold, the processing device 140 may determine the angle threshold as 10 degrees and the count of the one or more reference directions as 3. The curvature threshold may be determined based on the system default setting, or set manually by the user. For example, the curvature threshold may be 30 degree, 45 degrees, 60 degrees, 90 degrees, 120 degrees, etc.

In some embodiments, after the angle threshold and the count of the one or more reference directions are determined, the processing device 140 may determine the one or more reference directions based on the normal direction, the angle threshold, and the count of the one or more reference directions. For example, the processing device 140 may randomly determine the one or more reference directions within the angle threshold around the normal direction. As another example, the processing device 140 may determine the one or more reference directions within the angle threshold around the normal direction at a uniform space.

In some embodiments, the processing device 140 may determine the first correction thickness of the orthodontic appliance corresponding to the local point by weighted averaging the lengths of the components of the movement path along the normal direction and each reference direction. For example, the component of the movement path along the normal direction and the component of the movement path along each reference direction may have the same weighting value. As another example, the component of the movement path along the normal direction may have a first weighting value, and the component of the movement path along each reference direction may have a second weighting value. The first weighting value may be different from the second weighting value. As still another example, each of the component of the movement path along the normal direction and the component of the movement path along each reference direction may have a specific weighting value. In some embodiments, a weighting value corresponding to a component may be determined based on the system default setting, or set manually by the user.

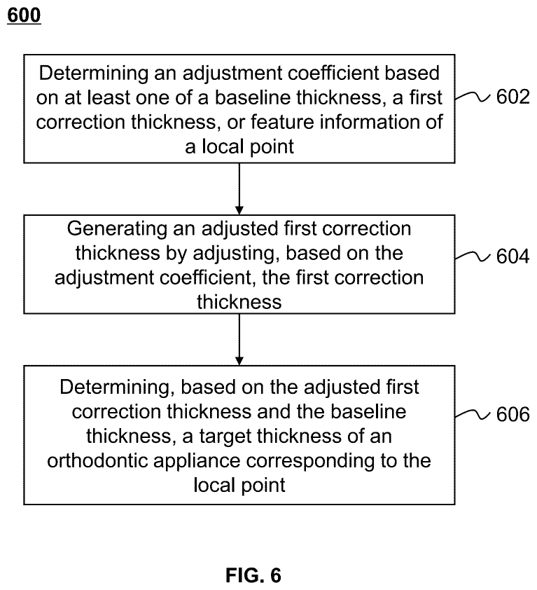

In some embodiments, the first correction thickness may be adjusted based on at least one of the baseline thickness, the first correction thickness, or feature information (also referred to as first feature information) of the local point. For example, the processing device 140 may determine an adjustment coefficient based on at least one of the baseline thickness, the first correction thickness, or the feature information of the local point, and generate an adjusted first correction thickness by adjusting the first correction thickness based on the adjustment coefficient. Further, the processing device 140 may determine the target thickness of the orthodontic appliance corresponding to the local point based on the adjusted first correction thickness and the baseline thickness. More descriptions regarding the determination of the target thickness based on the adjusted first correction thickness and the baseline thickness may be found elsewhere in the present disclosure (e.g., FIG. 6 and the descriptions thereof).

In some embodiments, the processing device 140 may obtain a location of a tooth corresponding to the local point, and determine a second correction thickness of the orthodontic appliance corresponding to the local point based on the location of the tooth. The processing device 140 may further determine the target thickness based on the second correction thickness, the first correction thickness (or the adjusted first correction thickness), and the baseline thickness. For example, the target thickness of the orthodontic appliance corresponding to the local point may be determined according to Equation (4):

$$T = {T}_{b} + {T}_{fc} + {T}_{sc} , \qquad ( 4 )$$

where T_{sc} refers to the second correction thickness of the orthodontic appliance corresponding to the local point.

The second correction thickness may be used for preventing posterior open bite. The posterior open bite is often caused as a result of the use of the orthodontic appliance (e.g., a clear aligner). The orthodontic appliance covers the occlusal of the posterior teeth, and the target bites down on the orthodontic appliance, which leads to retroclined anterior teeth (upper), or a lack of overjet. By introducing the second correction thickness at locations of the posterior teeth, the occurrence or the development of the posterior open bite can be avoided. In some embodiments, the second correction thickness may be determined for the posterior teeth (e.g., premolars and/or molars) of the target subject in order to realize the thickness increase towards the posterior teeth. For example, the processing device 140 may determine whether the tooth corresponding to the local point is one of the premolars and/or molars based on the location of the tooth. If the tooth corresponding to the local point is one of the premolars and/or molars, the processing device 140 may determine that the second correction thickness of the orthodontic appliance corresponding to the local point is greater than 0. In some embodiments, the second correction thickness may be determined based on the system default setting, or set manually by the user. For example, the second correction thickness may be within a range from 0.1 mm to 1.5 mm. As another example, the second correction thickness may be within a range from 0.5 mm to 1.2 mm. As still another example, the second correction thickness may be within a range from 0.8 mm to 1.1 mm. As yet another example, the second correction thickness may be 1.0 mm. In some embodiments, If the tooth corresponding to the local point is not one of the premolars and/or molars, the processing device 140 may determine that the second correction thickness of the orthodontic appliance corresponding to the local point is equal to 0 mm.

In some embodiments, the second correction thickness corresponding to each local point may be the same. For example, the second correction thickness corresponding to each local point may be 1.0 mm. In some embodiments, different local points may correspond to different second correction thicknesses. For example, the second correction thicknesses of a tooth may be increased in a continuous manner. For instance, a molar at a distal end may be 1 mm thicker than a molar at a mesial location, and the thickness change may be in a continuous manner instead of a step-wise manner.

In some embodiments, the processing device 140 may obtain a location of the local point, and determine a third correction thickness of the orthodontic appliance corresponding to the local point based on the location of the local point. The location of the local point may include a location of the local point on a corresponding tooth and a location of the tooth. The processing device 140 may further determine the target thickness based on the third correction thickness and at least one of the second correction thickness, the first correction thickness (or the adjusted first correction thickness), and the baseline thickness. For example, if the tooth is a posterior molar, and the local point is located at a lingual side of the tooth of the target subject, the processing device 140 may determine that the third correction thickness of the orthodontic appliance corresponding to the local point is greater than 0. In some embodiments, the third correction thickness may be determined based on the system default setting, or set manually by the user. For example, the third correction thickness may be within a range from 0.1 mm to 1.5 mm. As another example, the third correction thickness may be within a range from 0.5 mm to 1.2 mm. As still another example, the third correction thickness may be within a range from 0.8 mm to 1.1 mm. As yet another example, the third correction thickness may be 1.0 mm. In some embodiments, If the tooth corresponding to the local point is not one of the posterior molars, or the local point is not located at the lingual side of the tooth of the target subject, the processing device 140 may determine that the third correction thickness of the orthodontic appliance corresponding to the local point is equal to 0 mm.

By introducing the third correction thickness, focal points on the orthodontic appliance corresponding to at the lingual side of the posterior molars can be designed, which can provide forces to easily remove the orthodontic appliance from the teeth while ensuring the fit between the teeth and the orthodontic appliance, thereby improving the user experience and ensuring the orthodontic effectiveness. During a manufacturing process of an orthodontic appliance using a thermoforming technique (also referred to as a thermoformed orthodontic appliance), if the thermoformed orthodontic appliance fits perfectly onto a dental mold of the teeth of the target subject, it can become challenging to remove the thermoformed orthodontic appliance from the dental mold. In contrast, a directly printed orthodontic appliance does not encounter this issue, allowing for potentially superior fit. Moreover, a perfectly fitting orthodontic appliance may pose difficulties during removal when in use. Therefore, adjusting the thickness locally (e.g., by introducing the third correction thickness) can offer a solution to this problem. In addition, a portion of the orthodontic appliance corresponding to the lingual side of the posterior molars can be thickened, which can prevent deformation and fracture of the orthodontic appliance.

In some embodiments, the processing device 140 may determine a fourth correction thickness based on at least one preset requirement, and determine the target thickness based on the fourth correction thickness and at least one of the third correction thickness, the second correction thickness, the first correction thickness (or the adjusted first correction thickness), and the baseline thickness. For example, the processing device 140 may determine whether the target thickness of the orthodontic appliance corresponding to the local point is satisfied with the at least one preset requirement. The at least one preset requirements may include a production requirement, a treatment requirement, a structural requirement, or the like, or any combination thereof. For example, the treatment requirement may include whether the orthodontic appliance with the target thickness corresponding to each local point satisfies the orthodontic treatment plan. As another example, the structural requirement may include whether the orthodontic appliance with the target thickness corresponding to each local point fits with the teeth of the target subject. If the target thickness of the orthodontic appliance corresponding to the local point is satisfied with the preset requirements, the processing device 140 may determine that the fourth correction thickness of the orthodontic appliance corresponding to the local point is equal to 0. If the target thickness of the orthodontic appliance corresponding to the local point is not satisfied with the preset requirements, the processing device 140 may determine that the fourth correction thickness of the orthodontic appliance corresponding to the local point is greater than 0.

The fourth correction thickness may be used for adjusting the target thickness of the orthodontic appliance corresponding to the local point according to the preset requirements. In some embodiments, the fourth correction thickness may be determined based on the system default setting, or set manually by the user. For example, the fourth correction thickness may be within a range from 0.1 mm to 1.5 mm. As another example, the fourth correction thickness may be within a range from 0.5 mm to 1.2 mm. As still another example, the fourth correction thickness may be within a range from 0.8 mm to 1.1 mm. As yet another example, the fourth correction thickness may be 1.0 mm.

By introducing the fourth correction thickness, the target thickness of the orthodontic appliance corresponding to the local point can be adjusted, so that the orthodontic appliance to be produced can satisfy the preset requirements, thereby ensuring the accuracy of the orthodontic appliance and the orthodontic effectiveness.

In some embodiments, the processing device 140 may obtain a thickness determination model. The thickness determination model may be a trained machine learning model. The processing device 140 may determine the target thickness of the orthodontic appliance corresponding to the local point based on the movement path and the baseline thickness using the thickness determination model. For example, the processing device 140 may input the movement path and the baseline thickness into the thickness determination model, and the thickness determination model may output the target thickness of the orthodontic appliance corresponding to the local point.

As another example, the processing device 140 may sample feature points along the movement path, and determine the target thickness of the orthodontic appliance corresponding to the local point based on the feature points and the baseline thickness using the thickness determination model. The feature points may be used to characterize the movement path. For example, the feature points may include a starting point, a mid-point, an end point, or the like, or any combination thereof. The count of the feature points may be determined based on a smoothness of the teeth at the local point and/or the angle between the movement path of the local point and the normal direction of the local point. For example, the larger the smoothness of the teeth at the local point and/or the angle between the movement path of the local point and the normal direction of the local point, the larger the count of the feature points. The count of the feature points may be determined in a similar manner as how the count of the one or more reference directions is determined, which is not repeated herein.

The thickness determination model refers to a model used to determine the target thickness of the orthodontic appliance corresponding to the local point. In some embodiments, the classification model may be a trained machine learning model, such as, a neural network model, which is not limited to herein.

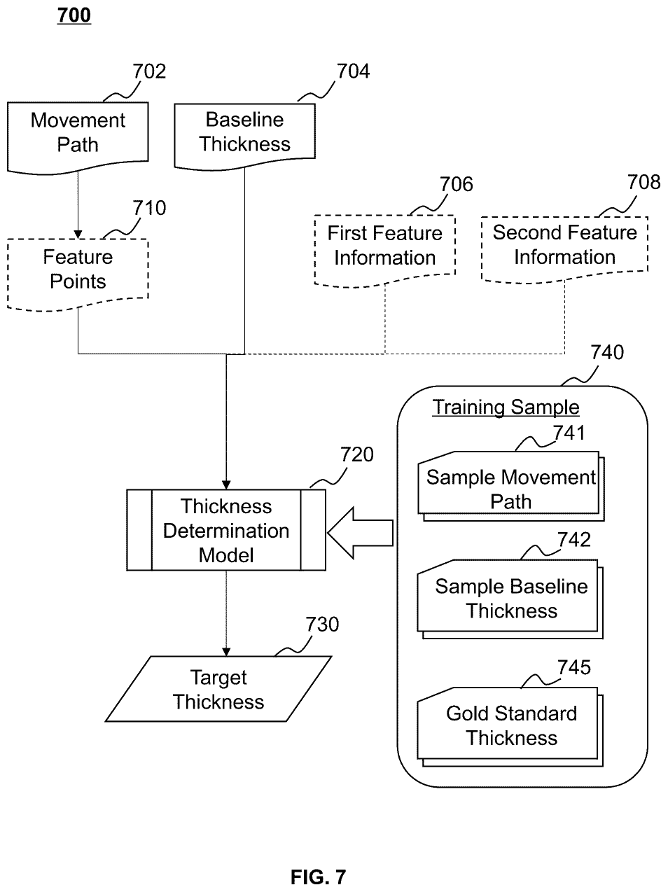

In some embodiments, the thickness determination model may be generated through a training process. For example, the processing device 140 may obtain a plurality of training samples. Each of the plurality of training samples may include a sample baseline thickness and a sample movement path of a sample local point, and a gold standard thickness corresponding to the sample local point. The gold standard thickness may indicate a sample thickness of a sample orthodontic appliance at the sample local point. The processing device 140 may generate the thickness determination model by training an initial model using the plurality of training samples. More descriptions regarding the thickness determination model may be found elsewhere in the present disclosure (e.g., FIG. 7 and the descriptions thereof).

In some embodiments, an input of the thickness determination model may further include the feature information of the local point and/or feature information (also referred to as second feature information) of one or more adjacent points of the local point. For example, the processing device 140 may input the movement path, the baseline thickness, the feature information of the local point, and the feature information of the one or more adjacent points of the local point into the thickness determination model, and the thickness determination model may output the target thickness of the orthodontic appliance corresponding to the local point. A count of the adjacent points may be determined in a similar manner as how the count of the one or more reference directions is determined, which is not repeated herein. Correspondingly, each of the plurality of training samples may further include sample feature information of the sample local point and/or sample feature information of one or more sample adjacent points of the sample local point.

In 308, the processing device 140 (e.g., the control module 240) may direct a 3D printer to integrally produce, based on the target thickness of the orthodontic appliance at each local point, the orthodontic appliance.

In some embodiments, the processing device 140 may obtain the preliminary digital model (e.g., the preliminary digital model 450) of the orthodontic appliance, and generate a target digital model of the orthodontic appliance based on the target thickness of the orthodontic appliance at each local point and the preliminary digital model. Further, the processing device 140 may direct the 3D printer (e.g., the 3D printer 110) to integrally produce the orthodontic appliance based on the target digital model. The preliminary digital model refers to a digital model of the orthodontic appliance with the baseline thickness, and the target digital model refers to a digital model of the orthodontic appliance with the target thickness at each local point. For example, the processing device 140 may generate the target digital model by adjusting the preliminary digital model based on the target thickness of the orthodontic appliance at each local point using the computer software, such as LuxCreo's LuxDesign.

In some embodiments, the processing device 140 may generate a processed digital model by post-processing the target digital model, and direct the 3D printer (e.g., the 3D printer 110) to integrally produce the orthodontic appliance based on the processed digital model. The post-processing may include trimming, polishing, smoothing, an anti-sensitive operation, or the like, or any combination thereof. In some embodiments, the processing device 140 may post-process the target digital model using the computer software, such as LuxCreo's LuxDesign.

In some embodiments, before the integral production of the orthodontic appliance, the processing device 140 may display the target digital model (or the processed digital model) to the user. For example, the target digital model (or the processed digital model) may be displayed through a user interface of the at least one terminal 130, and the user may confirm and/or adjust the target digital model (or the processed digital model) through an input device (e.g., a mouse, a keyboard, a touch screen, etc.) of the at least one terminal 130.

According to some embodiments of the present disclosure, by determining the target thickness of the orthodontic appliance at each local point, each local point of the orthodontic appliance can have a customized and ununiform thickness, which can provide customized forces to the teeth, thereby improving the orthodontic effectiveness. In addition, by determining the target thickness of the orthodontic appliance at each local point, the orthodontic appliance can be integrally produced, and no attachments or less attachments (e.g., attachments for improving gripping forces between the teeth and the orthodontic appliance, attachments for providing forces to easily remove the orthodontic appliance from the teeth, etc.) need to be disposed on the orthodontic appliance, which can simplify the orthodontic appliance production process, thereby improving the efficiency of the orthodontic appliance production. In addition, by introducing the first correction thickness and the second correction thickness to determine the target thickness of the orthodontic appliance at each local point, the feature information of the local points and/or the teeth can be considered, which can improve the accuracy of the thickness determination and the customization of the orthodontic appliance, thereby improving the orthodontic effectiveness.

FIG. 6 is a flowchart illustrating an exemplary process 600 for determining a target thickness of an orthodontic appliance corresponding to a local point according to some embodiments of the present disclosure. In some embodiments, the process 600 may be performed to achieve at least part of operation 306 as described in connection with FIG. 3.

In 602, the processing device 140 (e.g., the third determination module 230) may determine an adjustment coefficient based on at least one of a baseline thickness, a first correction thickness, or feature information of a local point.

The feature information may include appearance parameter(s) (e.g., a thickness, a curvature, a smoothness, a rigidity, etc.) of the tooth at the local point, a tolerance of a movement of the local point, an age of the target subject, a diagnostic status of the tooth where the local point is located (e.g., whether the tooth is loose, whether the tooth is a decayed tooth, a sensitivity degree, etc.), or the like, or any combination thereof.

In some embodiments, the adjustment coefficient may relate to the at least one of the baseline thickness, the first correction thickness, or the feature information of the local point. For example, if a rigidity of a tooth at a local point is relatively large, the adjustment coefficient corresponding to the local point may be relatively large. As another example, if a first local point is closer to the root of the teeth than a second local point (e.g., near the biting surface), the adjustment coefficient of the first local point may be larger than the adjustment coefficient of the second local point. Therefore, if a movement path of the first local point is equal to a movement path of the second local point (or a first correction thickness of the first local point is equal to a first correction thickness of the second local point), the orthodontic appliance corresponding to the first local point may provide a larger orthodontic force than the orthodontic appliance corresponding to the second local point. In these cases, the adjustment coefficient can be used to reflect differences between the feature information of the local points, which can provide customized and localized forces to different teeth or different local points on the teeth, thereby improving the orthodontic effectiveness.

In some embodiments, the processing device 140 may determine a corresponding relationship between adjustment coefficients, baseline thicknesses, first correction thicknesses, feature information of local points, and determine the adjustment coefficient based on the corresponding relationship and the at least one of the baseline thickness, the first correction thickness, or the feature information of the local point. For example, the processing device 140 may create a table, a graph, etc., indicating the corresponding relationship based on historical data. When the at least one of the baseline thickness, the first correction thickness, or the feature information of the local point is obtained, the processing device 140 may retrieve the table, the graph, etc., to determine the adjustment coefficient based on the at least one of the baseline thickness, the first correction thickness, or the feature information of the local point.

In 604, the processing device 140 (e.g., the third determination module 230) may generate an adjusted first correction thickness by adjusting the first correction thickness based on the adjustment coefficient.

For example, the adjusted first correction thickness may be generated by multiplying the first correction thickness with the adjustment coefficient.

In 606, the processing device 140 (e.g., the third determination module 230) may determine, based on the adjusted first correction thickness and the baseline thickness, the target thickness of the orthodontic appliance corresponding to the local point.

For example, the target thickness of the orthodontic appliance corresponding to the local point may be a sum of the adjusted first correction thickness and the baseline thickness. For example, the target thickness of the orthodontic appliance corresponding to the local point may be determined according to Equation (5):

$$T = {T}_{b} + {aT}_{fc} , \qquad ( 5 )$$

where a refers to the adjusted first correction thickness of the orthodontic appliance corresponding to the local point.

As another example, the processing device 140 may obtain a second correction thickness, and determine the target thickness by summing the second correction thickness, the adjusted first correction thickness, and the baseline thickness.

According to some embodiments of the present disclosure, by considering the at least one of the baseline thickness, the first correction thickness, or the feature information of the local point, the first correction thickness can be adjusted, which can improve the accuracy of the determination of the target thickness, thereby improving the accuracy of the orthodontic appliance and the orthodontic effectiveness.

FIG. 7 is a schematic diagram illustrating an exemplary process 700 for determining a target thickness of an orthodontic appliance at a local point using a thickness determination model according to some embodiments of the present disclosure.

As illustrated in FIG. 7, in some embodiments, a movement path 702 and a baseline thickness 704 corresponding to a local point may be input into a thickness determination model 720, and the thickness determination model 720 may output a target thickness 730.

In some embodiments, the thickness determination model 720 may be obtained by training an initial model based on a plurality of training samples 740. In some embodiments, each of the plurality of training samples 740 may include a sample movement path 741 and a sample baseline thickness 742 corresponding to a sample local point, and a gold standard thickness 745 corresponding to the sample local point. The gold standard thickness 745 may indicate a sample thickness of a sample orthodontic appliance at the sample local point.

The sample movement path 741 and the sample baseline thickness 742 corresponding to the sample local point may be obtained in a similar manner as how the movement path and the baseline thickness corresponding to the local point are obtained as described in FIG. 3, which is not repeated herein. The gold standard thickness 745 may be generated in a similar manner as how the target thickness corresponding to the local point is obtained as described in FIG. 3, which is not repeated herein. In some embodiments, the sample movement path 741, the sample baseline thickness 742, and the gold standard thickness 745 corresponding to the sample local point may be obtained based on historically produced orthodontic appliances.

In some embodiments, the thickness determination model 720 may be generated by training the initial model using the plurality of training samples 740. During the training process, parameter(s) of the initial model may be updated through one or more iterations.

For example, for each of the plurality of training samples 740, the processing device 140 may determine a predicted thickness by inputting the sample movement path 741 and the sample baseline thickness 742 corresponding to the sample local point of the training sample into the initial model. The processing device 140 may determine a training loss between the predicted thickness and the gold standard thickness 745 of the training sample, and update the parameter(s) of the initial model based on the training loss. For example, the processing device 140 may adjust the parameter(s) of the initial model using a backpropagation algorithm based on the training loss to reduce the difference between predicted thicknesses and the gold standard thicknesses, for example, by continuously adjusting the parameter(s) of the initial model to reduce or minimize the training loss.

In some embodiments, the training loss may include a perceptual loss, a squared loss, a logistic regression loss, a focal loss, a dice loss, or the like, or any combination thereof.

In some embodiments, feature points 710 may be sampled along the movement path 702, and information of the feature points 710 and the baseline thickness 704 may be input into the thickness determination model 720, and the thickness determination model 720 may output the target thickness 730.

In some embodiments, the input of the thickness determination model 720 may further include first feature information 706 of the local point and/or second feature information 708 of one or more adjacent points of the local point. For example, the movement path 702 (or the feature points 710), the baseline thickness 704, the first feature information 706, and the second feature information 708 may be input into the thickness determination model 720, and the thickness determination model 720 may output the target thickness 730 of the orthodontic appliance corresponding to the local point. Correspondingly, each of the plurality of training samples 740 may further include sample feature information of the sample local point and/or sample feature information of one or more sample adjacent points of the sample local point (not shown).

According to some embodiments of the present disclosure, the target thickness of the orthodontic appliance corresponding to the local point can be automatically determined using the thickness determination model, which can improve the efficiency and accuracy of the determination of the target thickness, thereby improving the efficiency and accuracy of the orthodontic appliance production.

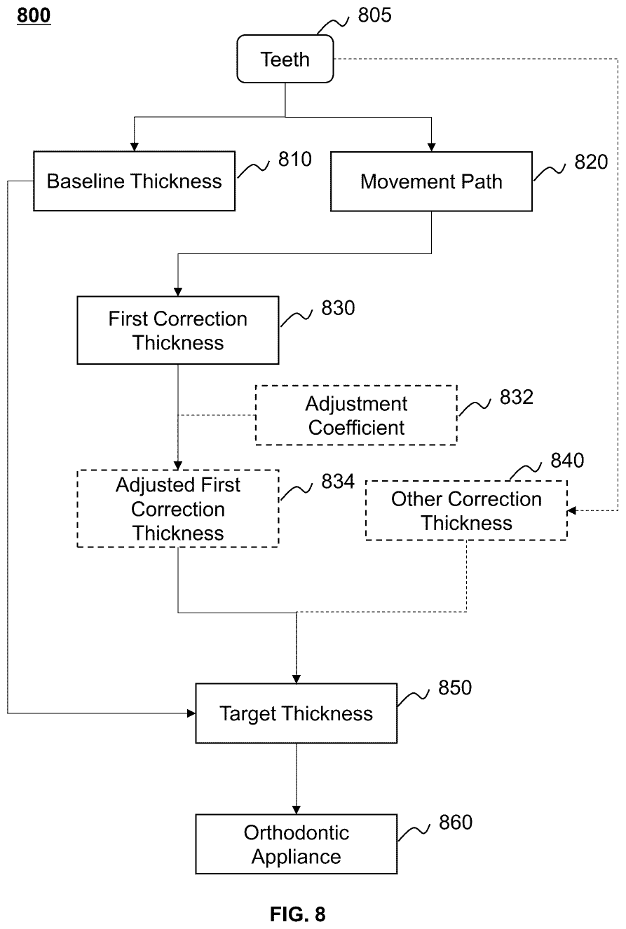

FIG. 8 is a schematic diagram illustrating an exemplary process 800 for orthodontic appliance production according to some embodiments of the present disclosure.

As shown in FIG. 8, a baseline thickness 810 of an orthodontic appliance 860 for orthodontically treating teeth 805 of a target subject may be determined. For each local point on the teeth 805 of the target subject, a movement path 820 of the local point from an initial location of the local point to a target location of the local point may be determined. A first correction thickness 830 of the orthodontic appliance 860 corresponding to the local point may be determined based on the movement path 820. A target thickness 850 of the orthodontic appliance 860 at the local point may be determined based on the first correction thickness 830 and the baseline thickness 810. The orthodontic appliance 860 may be integrally produced based on the target thickness 850 of the orthodontic appliance 860 at each local point through a 3D printer (e.g., the 3D printer 110).

In some embodiments, an adjusted first correction thickness 834 of the orthodontic appliance 860 at the local point may be determined by adjusting the first correction thickness 830 based on an adjustment coefficient 832, wherein the adjustment coefficient 832 may be determined based on at least one of the baseline thickness 810, the first correction thickness 830, or feature information (not shown) of the local point. Therefore, the target thickness 850 of the orthodontic appliance 860 at the local point may be determined based on the adjusted first correction thickness 834 and the baseline thickness 810.

In some embodiments, at least one other correction thickness (e.g., a second correction thickness, a third correction thickness, a fourth correction thickness, etc.) 840 may be determined based on information of the teeth 805 (e.g., a location of a tooth corresponding to the local point, a location of the local point on the corresponding tooth). Correspondingly, the target thickness 850 of the orthodontic appliance 860 at the local point may be determined based on the at least one other correction thickness 840, the first correction thickness 830 (or the adjusted first correction thickness 834), and the baseline thickness 810.

Processes 300 and 600-800 may be implemented in the system 100 illustrated in FIG. 1. For example, the processes 300 and 600-800 may be stored in the storage device 150 as a form of instructions, and invoked and/or executed by the processing device 140. The operations of the illustrated process presented below are intended to be illustrative. In some embodiments, the processes 300 and 600-800 may be accomplished with one or more additional operations not described, and/or without one or more of the operations discussed. Additionally, the order in which the operations of the processes 300 and 600-800 are not intended to be limiting.

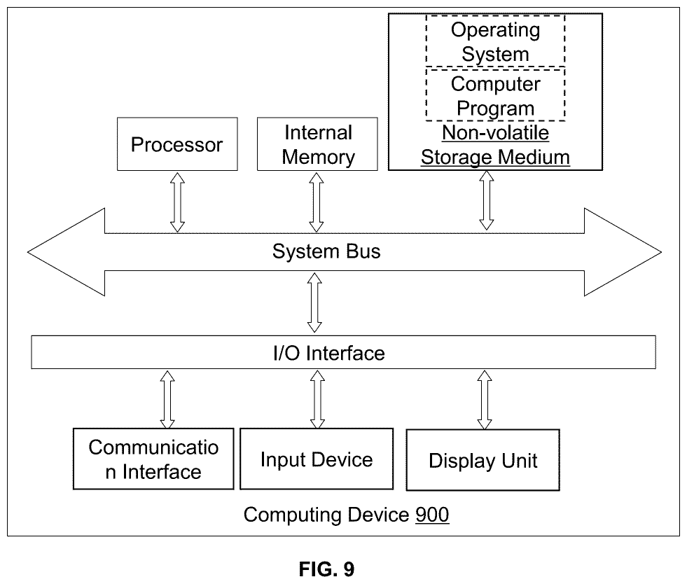

FIG. 9 is a schematic diagram illustrating an exemplary computing device 900 according to some embodiments of the present disclosure.

In some embodiments, one or more components of the system 100 for orthodontic appliance production may be implemented on the computing device 900. For example, the processing device 140 may be implemented on the computing device 900 and configured to implement the functions and/or methods disclosed in the present disclosure.

The computing device 900 may include any components used to implement the system 100 described in the present disclosure. For example, the processing device 140 may be implemented through hardware, software program, firmware, or any combination thereof, on the computing device 900. For illustration purposes, only one computer is described in FIG. 9, but computing functions related to the system 100 described in the present disclosure may be implemented in a distributed fashion by a group of similar platforms to spread the processing load of the system 100.

The computing device 900 may include a communication port connected to a network to achieve data communication. The computing device 900 may include a processor (e.g., a central processing unit (CPU)), a memory, a communication interface, a display unit, and an input device connected by a system bus. The processor of the computing device 900 may be used to provide computing and control capabilities. The memory of the computing device 900 may include a non-volatile storage medium, an internal memory. The non-volatile storage medium may store an operating system and a computer program. The internal memory may provide an environment for the execution of the operating system and the computer program in the non-volatile storage medium. The communication interface of the computing device 900 may be used for wired or wireless communication with an external terminal. The wireless communication may be realized through Wi-Fi, a mobile cellular network, a near field communication (NFC), etc. When the computer program is executed by the processor, a method for determining feature points may be implemented. The display unit of the computing device 900 may include a liquid crystal display screen or an electronic ink display screen. The input device of the computing device 900 may include a touch layer covered on the display unit, a device (e.g., a button, a trackball, a touchpad, etc.) set on the housing of the computing device 900, an external keyboard, an external trackpad, an external mouse, etc.

Merely for illustration, only one processor is described in FIG. 9. However, it should be noted that the computing device 900 in the present disclosure may also include multiple processors. Thus operations and/or method steps that are performed by one processor as described in the present disclosure may also be jointly or separately performed by the multiple processors. For example, if the processor of the computing device 900 in the present disclosure executes both operation A and operation B, it should be understood that operation A and operation B may also be performed by two or more different processors jointly or separately (e.g., a first processor executes operation A and a second processor executes operation B, or the first and second processors jointly execute operations A and B).

Some embodiments of the present disclosure also provide a computer-readable storage medium. The computer-readable storage medium may store computer-executable instructions, and the computer-executable instructions may be used to cause a computer to implement the processes in the above embodiments of the present disclosure.

Having thus described the basic concepts, it may be rather apparent to those skilled in the art after reading this detailed disclosure that the foregoing detailed disclosure is intended to be presented by way of example only and is not limiting. Various alterations, improvements, and modifications may occur and are intended for those skilled in the art, though not expressly stated herein. These alterations, improvements, and modifications are intended to be suggested by this disclosure, and are within the spirit and scope of the exemplary embodiments of this disclosure.

Moreover, certain terminology has been used to describe embodiments of the present disclosure. For example, the terms “one embodiment,” “an embodiment,” and/or “some embodiments” mean that a particular feature, structure, or characteristic described in connection with the embodiment is included in at least one embodiment of the present disclosure. Therefore, it is emphasized and should be appreciated that two or more references to “an embodiment” or “one embodiment” or “an alternative embodiment” in various portions of this disclosure are not necessarily all referring to the same embodiment. Furthermore, the particular features, structures, or characteristics may be combined as suitable in one or more embodiments of the present disclosure.

Furthermore, the recited order of processing elements or sequences, or the use of numbers, letters, or other designations therefore, is not intended to limit the claimed processes and methods to any order except as may be specified in the claims. Although the above disclosure discusses through various examples what is currently considered to be a variety of useful embodiments of the disclosure, it is to be understood that such detail is solely for that purpose, and that the appended claims are not limited to the disclosed embodiments, but, on the contrary, are intended to cover modifications and equivalent arrangements that are within the spirit and scope of the disclosed embodiments. For example, although the implementation of various components described above may be embodied in a hardware device, it may also be implemented as a software only solution, e.g., an installation on an existing server or mobile device.

Similarly, it should be appreciated that in the foregoing description of embodiments of the present disclosure, various features are sometimes grouped together in a single embodiment, figure, or description thereof for the purpose of streamlining the disclosure aiding in the understanding of one or more of the various inventive embodiments. This method of disclosure, however, is not to be interpreted as reflecting an intention that the claimed subject matter requires more features than are expressly recited in each claim. Rather, inventive embodiments lie in less than all features of a single foregoing disclosed embodiment.

In some embodiments, the numbers expressing quantities or properties used to describe and claim certain embodiments of the application are to be understood as being modified in some instances by the term “about,” “approximate,” or “substantially.” For example, “about,” “approximate,” or “substantially” may indicate ±20% variation of the value it describes, unless otherwise stated. Accordingly, in some embodiments, the numerical parameters set forth in the written description and attached claims are approximations that may vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the application are approximations, the numerical values set forth in the specific examples are reported as precisely as practicable.

Each of the patents, patent applications, publications of patent applications, and other material, such as articles, books, specifications, publications, documents, things, and/or the like, referenced herein is hereby incorporated herein by this reference in its entirety for all purposes, excepting any prosecution file history associated with same, any of same that is inconsistent with or in conflict with the present document, or any of same that may have a limiting effect as to the broadest scope of the claims now or later associated with the present document. By way of example, should there be any inconsistency or conflict between the description, definition, and/or the use of a term associated with any of the incorporated material and that associated with the present document, the description, definition, and/or the use of the term in the present document shall prevail.

In closing, it is to be understood that the embodiments of the application disclosed herein are illustrative of the principles of the embodiments of the application. Other modifications that may be employed may be within the scope of the application. Thus, by way of example, but not of limitation, alternative configurations of the embodiments of the application may be utilized in accordance with the teachings herein. Accordingly, embodiments of the present application are not limited to that precisely as shown and described.

1. A method for orthodontic appliance production, implemented on a computing device having at least one processor and at least one storage device, the method comprising:
determining a baseline thickness of an orthodontic appliance for orthodontically treating teeth of a target subject;
for each local point on the teeth of the target subject,
determining a movement path of the local point from an initial location of the local point to a target location of the local point; and
determining, based on the movement path and the baseline thickness, a target thickness of the orthodontic appliance corresponding to the local point; and
directing a three-dimensional (3D) printer to integrally produce, based on the target thickness of the orthodontic appliance at each local point, the orthodontic appliance.

2. The method of claim 1, wherein the determining, based on the movement path and the baseline thickness, a target thickness of the orthodontic appliance corresponding to the local point includes:
determining, based on the movement path, a first correction thickness of the orthodontic appliance corresponding to the local point, the first correction thickness relating to a component of the movement path along a normal direction of the local point; and
determining, based on the first correction thickness and the baseline thickness, the target thickness of the orthodontic appliance corresponding to the local point.

3. The method of claim 2, wherein the determining, based on the first correction thickness and the baseline thickness, the target thickness of the orthodontic appliance corresponding to the local point includes:
determining an adjustment coefficient based on at least one of the baseline thickness, the first correction thickness, or feature information of the local point;
generating an adjusted first correction thickness by adjusting, based on the adjustment coefficient, the first correction thickness; and
determining, based on the adjusted first correction thickness and the baseline thickness, the target thickness of the orthodontic appliance corresponding to the local point.

4. The method of claim 2, wherein the determining, based on the first correction thickness and the baseline thickness, the target thickness of the orthodontic appliance corresponding to the local point includes:
obtaining a location of a tooth corresponding to the local point;
determining, based on the location of the tooth, a second correction thickness of the orthodontic appliance corresponding to the local point; and
determining the target thickness based on the second correction thickness, the first correction thickness, and the baseline thickness.

5. The method of claim 4, wherein the determining the target thickness based on the second correction thickness, the first correction thickness, and the baseline thickness includes:
obtaining a location of the local point;
determining, based on the location of the local point, a third correction thickness of the orthodontic appliance corresponding to the local point, the third correction thickness being configured to provide focal points on the orthodontic appliance to remove the orthodontic appliance from the teeth; and
determining the target thickness based on the third correction thickness, the second correction thickness, the first correction thickness, and the baseline thickness.

6. The method of claim 4, wherein the determining the target thickness based on the second correction thickness, the first correction thickness, and the baseline thickness includes:
determining, based on at least one preset requirement, a fourth correction thickness; and
determining the target thickness based on the fourth correction thickness, the second correction thickness, the first correction thickness, and the baseline thickness.

7. The method of claim 2, wherein the determining, based on the movement path, a first correction thickness of the orthodontic appliance corresponding to the local point includes:
determining, based on the normal direction of the local point, one or more reference directions;
for each of the one or more reference directions, determining a component of the movement path along the direction; and
determining, based on the component of the movement path along the normal direction and the component of the movement path along each reference direction, the first correction thickness of the orthodontic appliance corresponding to the local point.

8. The method of claim 7, wherein the one or more reference directions are determined further based on a curvature of the teeth at the local point and/or an angle between the movement path of the local point and the normal direction of the local point.

9. The method of claim 1, wherein the determining, based on the movement path and the baseline thickness, a target thickness of the orthodontic appliance corresponding to the local point includes:
obtaining a thickness determination model, the thickness determination model being a trained machine learning model; and
determining the target thickness of the orthodontic appliance corresponding to the local point based on the movement path and the baseline thickness using the thickness determination model.

10. The method of claim 9, wherein the determining the target thickness of the orthodontic appliance corresponding to the local point based on the movement path and the baseline thickness using the thickness determination model comprises:
sampling feature points along the movement path, wherein the count of the feature points is determined based on a smoothness of the teeth at the local point and/or an angle between the movement path of the local point and the normal direction of the local point;
determining the target thickness of the orthodontic appliance corresponding to the local point based on the feature points and the baseline thickness using the thickness determination model.

11. The method of claim 10, wherein an input of the thickness determination model further includes feature information of the local point and/or feature information of one or more adjacent points of the local point.

12. The method of claim 1, wherein the directing a 3D printer to integrally produce, based on the target thickness of the orthodontic appliance at each local point, the orthodontic appliance includes:
obtaining a preliminary digital model of the orthodontic appliance;
generating, based on the target thickness of the orthodontic appliance at each local point and the preliminary digital model, a target digital model of the orthodontic appliance; and
directing the 3D printer to integrally produce, based on the target digital model, the orthodontic appliance.

13. The method of claim 12, wherein the directing 3D printer to integrally produce, based on the target digital model, the orthodontic appliance includes:
generating a processed digital model by post-processing the target digital model, the post-processing including at least one of trimming and polishing; and
directing the 3D printer to integrally produce, based on the processed digital model, the orthodontic appliance.

14. A system for orthodontic appliance production, comprising:
at least one storage device including a set of instructions; and
at least one processor configured to communicate with the at least one storage device, wherein when executing the set of instructions, the at least one processor is configured to direct the system to perform operations including:
determining a baseline thickness of an orthodontic appliance for orthodontically treating teeth of a target subject;
for each local point on the teeth of the target subject,
determining a movement path of the local point from an initial location of the local point to a target location of the local point; and
determining, based on the movement path and the baseline thickness, a target thickness of the orthodontic appliance corresponding to the local point; and
directing a three-dimensional (3D) printer to integrally produce, based on the target thickness of the orthodontic appliance at each local point, the orthodontic appliance.

15. The system of claim 14, wherein the determining, based on the movement path and the baseline thickness, a target thickness of the orthodontic appliance corresponding to the local point includes:
determining, based on the movement path, a first correction thickness of the orthodontic appliance corresponding to the local point, the first correction thickness relating to a component of the movement path along a normal direction of the local point; and
determining, based on the first correction thickness and the baseline thickness, the target thickness of the orthodontic appliance corresponding to the local point.

16. The system of claim 15, wherein the determining, based on the first correction thickness and the baseline thickness, the target thickness of the orthodontic appliance corresponding to the local point includes:
determining an adjustment coefficient based on at least one of the baseline thickness, the first correction thickness, or feature information of the local point;
generating an adjusted first correction thickness by adjusting, based on the adjustment coefficient, the first correction thickness; and
determining, based on the adjusted first correction thickness and the baseline thickness, the target thickness of the orthodontic appliance corresponding to the local point.

17-19. (Canceled)

20. The system of claim 15, wherein the determining, based on the movement path, a first correction thickness of the orthodontic appliance corresponding to the local point includes:
determining, based on the normal direction of the local point, one or more reference directions;
for each of the one or more reference directions, determining a component of the movement path along the direction; and
determining, based on the component of the movement path along the normal direction and the component of the movement path along each reference direction, the first correction thickness of the orthodontic appliance corresponding to the local point.

21. (Canceled)

22. The system of claim 14, wherein the determining, based on the movement path and the baseline thickness, a target thickness of the orthodontic appliance corresponding to the local point includes:
obtaining a thickness determination model, the thickness determination model being a trained machine learning model; and
determining the target thickness of the orthodontic appliance corresponding to the local point based on the movement path and the baseline thickness using the thickness determination model.

23-24. (Canceled)

25. The system of claim 14, wherein the directing a 3D printer to integrally produce, based on the target thickness of the orthodontic appliance at each local point, the orthodontic appliance includes:
obtaining a preliminary digital model of the orthodontic appliance;
generating, based on the target thickness of the orthodontic appliance at each local point and the preliminary digital model, a target digital model of the orthodontic appliance; and
directing the 3D printer to integrally produce, based on the target digital model, the orthodontic appliance.

26. (Canceled)

27. A non-transitory computer readable medium, comprising executable instructions that, when executed by at least one processor, direct the at least one processor to perform a method for orthodontic appliance production, the method comprising:
determining a baseline thickness of an orthodontic appliance for orthodontically treating teeth of a target subject;
for each local point on the teeth of the target subject,
determining a movement path of the local point from an initial location of the local point to a target location of the local point; and
determining, based on the movement path and the baseline thickness, a target thickness of the orthodontic appliance corresponding to the local point; and
directing a three-dimensional (3D) printer to integrally produce, based on the target thickness of the orthodontic appliance at each local point, the orthodontic appliance.
